(19) United States
(12) Patent Application Publication
(10) Pub. No.: US 2011/0082150 A1
(43) Pub. Date: Apr. 7, 2011

(54) Headache pre-emption by dihydroergotamine treatment during headache Precursor events

(76) Inventors: Robert Owen Cook, Hillsborough, NJ (US); Paul L. Durham, Nixa, MO (US); Shashidhar Kori, Dublin, CA (US); Thomas A. Armer, Cupertino, CA (US)

(21) Appl. No.: 12/592,287

(22) Filed: Nov. 19, 2009

Related U.S. Application Data
(63) Continuation-in-part of application No. 12/069,667, filed on Feb. 11, 2008.
Application No. 12/069,667 is a continuation-in-part of application No. 12/584,395, filed on Sep. 3, 2009.

(51) Int. Cl. A61K 31/4985; A61P 29/00; A61P 25/06

(52) U.S. Cl. 514/250

(57) ABSTRACT

Disclosed are methods that address providing a subject experiencing, or who has experienced, a headache precursor event and administering dihydroergotamine, or a pharmaceutically acceptable salt or complex thereof, to the subject by oral inhalation, in an amount effective to pre-empt a subsequent headache in the subject. Also disclosed are compositions that are related to those methods.

### CROSS REFERENCE TO RELATED CASES

This application is a continuation in part of U.S. patent application Ser. No. 12/069,667, filed on 11 Feb. 2008, and a continuation-in-part of U.S. patent application Ser. No. 12/584,395 filed on 3 Sep. 2009, the content of both of which are herein incorporated by reference in their entirety.

### TECHNICAL FIELD OF THE INVENTION

The invention relates to treatments and compositions for pre-empting headaches, and more particularly to treating subjects experiencing headache precursor events with DHE to pre-empt subsequent headaches.

### BACKGROUND OF THE INVENTION

Headache is a fairly common indication that ranges in severity from fairly mild and transitory to dehabilitating and chronic in duration. Headaches can have significant impact on individuals and society in aggregate.

Severe headaches, such as migraine, can be fairly common. For instance acute migraine affects approximately 13% of the population, predominately in females. See R B Lipton et al. “Migraine in the United States: a review of epidemiology and health care use.” Neurology 43 (6 Suppl 3): S6-10 (1993); B K Rasmussen et al. (1992). “Migraine with aura and migraine without aura: an epidemiological study.” Cephalalgia 12 (4): 221-8 (1992); T J Steiner et al. “The prevalence and disability burden of adult migraine in England and their relationships to age, gender and ethnicity”. Cephalalgia 23 (7): 519-27. (2003); M E Bigal et al. “Age-dependent prevalence and clinical features of migraine”. Neurology 67 (2): 246-51 (2006).

Improved headache treatments are needed urgently because of concerns regarding treatments for severe headaches. For instance, less than 30% of migraine sufferers report that they are very satisfied with their usual migraine treatment, and nearly two thirds of migraine sufferers experience unwanted side effects from antimigraine treatment. R M Gallagher et al., “Migraine: Diagnosis, Management, and New Treatment Options” Am J Manag Care 8:S58-S73 (2002).

Accordingly, methods and compositions that address the problems noted above and in the art are needed.

### SUMMARY OF THE INVENTION

In an aspect, the invention relates to a method comprising providing a subject experiencing a headache precursor event; and administering dihydroergotamine, or a pharmaceutically acceptable salt or complex thereof, to the subject by oral inhalation, in an amount effective to pre-empt a subsequent headache in the subject.

In another aspect, the invention relates to a method comprising providing a subject experiencing a headache precursor event, or who has experienced a headache precursor event within a previous period; and administering dihydroergotamine, or a pharmaceutically acceptable salt or complex thereof, to the subject by oral inhalation, in an amount effective to pre-empt a subsequent headache in the subject.

### BRIEF DESCRIPTION OF THE DRAWINGS

FIG. 1 shows percentage of subjects obtaining relief from pain with DHE versus placebo.

FIG. 2 shows pharmacokinetic profiles for achieving pain relief with minimal side effects.

FIG. 3 shows radioligand receptor binding profile for serotonergic receptor subtypes based on dose and administration route. Less than 20% was classed as inactive binding. “(h)” represents cloned human receptor subtypes.

FIG. 4 shows radioligand receptor binding profile for adrenergic and dopaminergic receptor subtypes based on dose and administration route. Less than 20% was classed as inactive binding. “(h)” represents cloned human receptor subtypes and “NS” indicates non-specific binding.

FIG. 5 shows selective agonism at 5-HT_{1B} and 5-HT_{2B} receptors at various concentrations of DHE.

FIG. 6 shows a plot of the geometric means of 8-OH DHE concentrations over time following administration of DHE by inhalation and intravenous (IV) routes.

FIG. 7 shows the effect of DHE or Sumatriptan on basal CGRP secretion levels.

FIG. 8 shows DHE or Sumatriptan repression of KCl-stimulated release.

FIG. 9 shows repression by DHE or Sumatriptan on capsaicin-stimulated release and that DHE does not significantly repress capsaicin-stimulated CGRP release.

FIG. 10 shows increase in MAP kinase phosphatase-1 (MKP-1) in DHE-treated trigeminal ganglia neurons.

FIG. 11 shows DHE-induced repression of p38 MAP kinase 14 levels in trigeminal ganglion neurons treated with vehicle (left panel), capsaicin (center panel), or capsaicin and DHE (right panel).

FIG. 12 shows decreased dye coupling (TRUEBLUE stain) between satellite glial cells and trigeminal ganglia neurons treated with either capsaicin (left panel) or with capsaicin and DHE (right panel).

FIG. 13 shows DHE-induced repression of connexin 26 levels in trigeminal ganglion neurons and satellite glia.

FIG. 14 shows expression of 5-HT₁ receptors in cultured trigeminal ganglion neurons: Row A: 5-HT_{1B}, 5-HT_{1D}, 5-HT_{1F}, and 5-HT_{1B}/5-HT_{1D}/5-HT_{1F} co-stain; Row B: β-tubulin.

FIG. 15 shows DHE increases expression of MAP kinase phosphatases (MKPs) in trigeminal ganglion neurons and satellite glial cell in vivo. Upper row: MKP stain; center row: DAPI stain; lower row: merged MKP/DAPI stain images; first panels: control vehicle and non-specific Ab; second panels: MKP-1 Ab; third panels: control vehicle and non-specific Ab; fourth panels: MKP-2 Ab; fifth panels: control vehicle and non-specific Ab; sixth panels: MKP-3 Ab.

FIG. 16 illustrates a scenario by which DHE can exert effects at multiple targets during the prodrome phase of migraine.

### DETAILED DESCRIPTION OF THE INVENTION

Introduction

The inventors have found, surprisingly, that the problems noted in the art can be addressed by providing methods, along with related compositions, that comprise providing a subject experiencing a headache precursor event; and administering dihydroergotamine, or a pharmaceutically acceptable salt or complex thereof, to the subject by oral inhalation, in an amount effective to pre-empt a subsequent headache in the subject. The problems in the art can be further addressed by providing methods, along with related compositions, that comprise providing a subject experiencing a headache precursor event, or who has experienced a headache precursor event within a previous period; and administering dihydroergotamine, or a pharmaceutically acceptable salt or complex thereof, to the subject by oral inhalation, in an amount effective to pre-empt a subsequent headache in the subject.

In particular, the inventors noted that certain subjects suffering from severe headache experience headache precursor events in advance of the severe headache. As discussed further below, such headache precursor events can comprise prodrome symptoms, premonitory symptoms, aura prior to headache onset, initial headache in a headache cluster, and headache trigger events. It is surprising that oral inhalation of DHE can provide an effective treatment to pre-empt a subsequent headache that occurs subsequent to a headache precursor event. It is even more surprising that oral inhalation of DHE can provide an effective treatment to pre-empt a subsequent headache while demonstrating a significant reduction in adverse events as compared to administration via other routes (such as intravenous administration). Reduction in adverse events while dosing DHE in amounts effective to pre-empt a subsequent headache is significant because the intensity of adverse events (such as nausea and vomiting) associated with conventional routes of dosing DHE have effectively precluded development of DHE as a treatment for the pre-emption of headaches. DHE conventionally administered intravenously (to ensure efficacious DHE concentration), or by intranasal administration (at efficacious exposure levels), results in such severe side effects that few subjects when presenting with headache precursor events have been willing to take conventional DHE dosage forms. This is because such conventional DHE dosage forms would inflict similar severe adverse events (nausea and vomiting) to those of the headaches the subject was hoping to pre-empt.

Reduction in adverse events via the oral inhalation route is demonstrated, among other places, in the data presented in Table 1. The data show, for instance, that inhaled DHE reduces incidence of nausea compared to intravenous administration (8% vs. 63% respectively). The exact mechanism by which inhaled DHE reduces adverse events compared to other routes of administration is unknown. However, various patterns of receptor binding and pharmacokinetic parameters, as set forth in more detail in the Examples, may provide some insight. Again, reduction in adverse events is useful because it makes administration of DHE clinically viable as a treatment for pre-emption of headache, whereas it was not clinically viable previously due to the intensity of associated adverse events and the complexities of intravenous administration.

Evidence of DHE's efficacy in pre-emption of headaches when administered to a subject experiencing a headache precursor event can be seen in the Examples, along with suggestive literature data obtained when DHE was administered by routes other than oral inhalation.

For instance, DHE, when administered by the intravenous route, is indicated for treatment of cluster headache once the first headache in a cluster has begun. See D.H.E. 45® product label. Also see J Olesen et al. eds. The Headaches, 2nd edn. Philadelphia: Lippincott Williams & Wilkins 2000:803. It is useful to note that intranasally administered DHE, in the form of MIGRANAL®, is not so indicated. While not wishing to be bound by a particular rationale, the inventors hypothesize that administration by the intravenous route provides sufficient DHE exposure to pre-empt subsequent cluster headaches, while administration by the intranasal route in the form of MIGRANAL® may provide insufficient exposure to pre-empt subsequent cluster headaches. In contrast, administration of DHE by oral inhalation provides sufficient drug exposure to be comparable to drug levels achieved by intravenous administration, thus supporting a reasonable expectation that orally inhaled DHE could be used to pre-empt subsequent cluster headaches once a headache precursor event (the first headache in a cluster) has begun.

In another instance, a single trial of DHE nasal spray during migraine prodrome (a headache precursor event) demonstrated statistically significant superiority over placebo at pre-empting the subsequent migraine. See S. Silberstein et al. eds., Wolff's Headache and Other Head Pain at 148 (7^{th} Edition) (2001). Although adverse events such as nausea and vomiting were not noted in this reference, presumably they would have significant as have been seen in other instances of intranasal administration at efficacious doses. A frequent side effect of dihydroergotamine is nausea for both iv and intranasal administration. J Olesen et al. eds., The Headaches (2^{nd} Edition) 464 (2000). Concomitant administration of an anti-emetic is recommended at least for the intravenous route. Id.

Further, the inventors have noted the following Experimental data, which is supportive of the efficacy of the inventive methods and compositions. While not wishing to be bound by particular mechanisms, the inventors note the following.

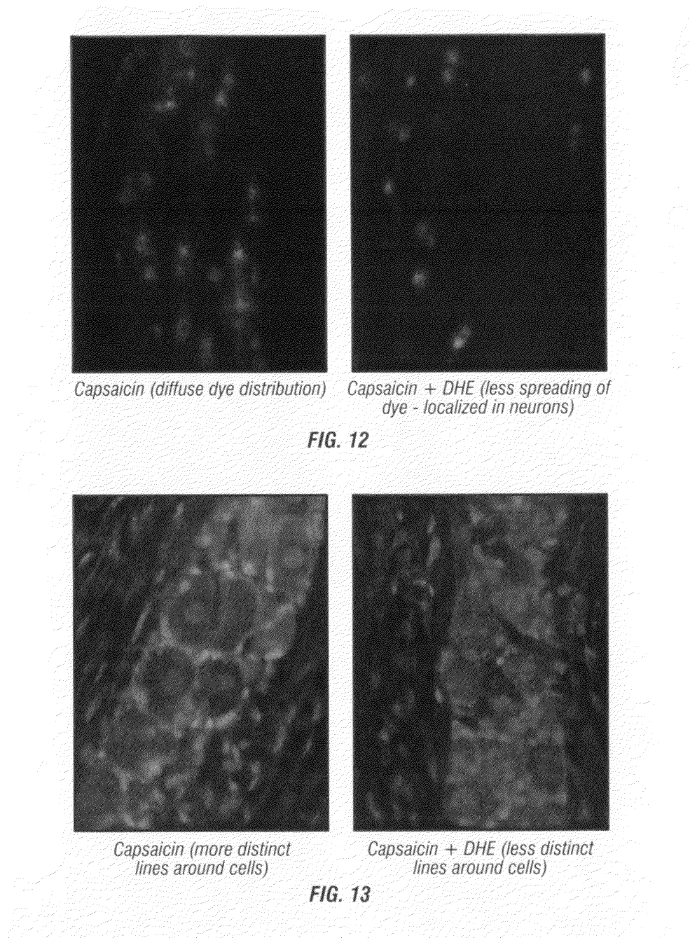

DHE appears to block inter-cellular transport via gap-junctions, in particular, perhaps by (i) binding to the gap-junction complex, thereby blocking the channel, (ii) by blocking translation/transcription of new connexin 26, a component of gap-junctions, thereby reducing the number of potential gap-junctions that may be created, (iii) both mechanisms (i) and (ii), or (iv) by another mechanism that involves 5-HT₁ receptor interactions with gap-junction formation/activation, via additional signal transduction pathway(s). As shown in FIGS. 12 and 13, DHE represses diffusion of TRUEBLUE dye between trigeminal ganglial cells and decreases the levels of connexin 26 in the cell surface membranes those cells.

This also suggests that recruitment of connexin 26 to the gap junction might be modulated by DHE and that upstream regulators of connexin induction might be affected or acted upon by DHE.

This disruption in neuronal communication or transmission between trigeminal neurons and glial cells, presumed to be occurring during a headache precursor event, could operate to pre-empt a subsequent headache severity, associated side effects and recurrence.

In another analysis of efficacy, and again continuing to not wish to be bound by a specific mechanism, the inventors note that activation of transport activity through gap junctions might be mediated by phosphorylation of connexin at tyrosine and serine/threonine residues by a number of protein kinases, including, but not limited to, casein kinase 1, c-SRC, MAP kinases ERK5 and ERK1/2, as well as the presence of increased intracellular [Ca²⁺. These pathways are in turn presumed to be activated by, for example, inflammatory cytokine (or lysophosphatidic acid) binding to receptors having tyrosine kinase activity that proceed to induce a cascade of further tyrosine kinase activities that activate downstream mixed tyrosine kinase and serine/threonine protein kinases such as MAPKKK and MAPKK. In contrast, the majority of neurotransmitters, such as serotonin, glutamate, dopamine, and noradrenalin, are believed to act via GPCRs and follow only serine/threonine protein kinase pathways, such as PK-A, PK-C. Interestingly, NO, which induces expression of CGRP, acts via another serine/threonine kinase, PK-G.

One modulator of MAP kinase activity is MAP kinase phosphatase, which is known to dephosphorylate MAP kinase, thereby inactivating the enzyme. This would result in reducing phosphorylation of connexins and result in reduced gap junction formation.

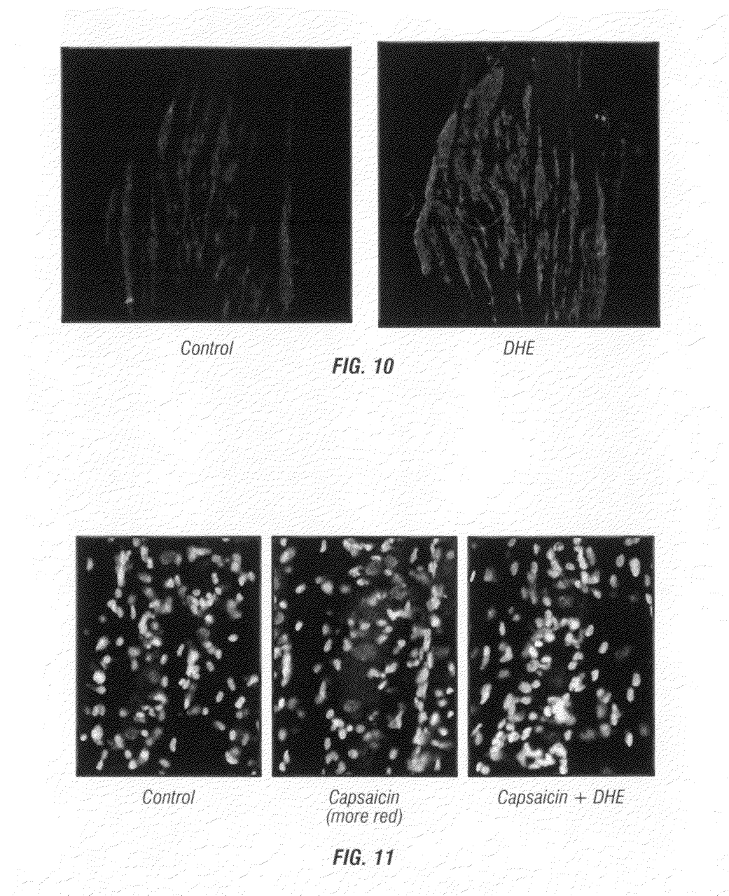

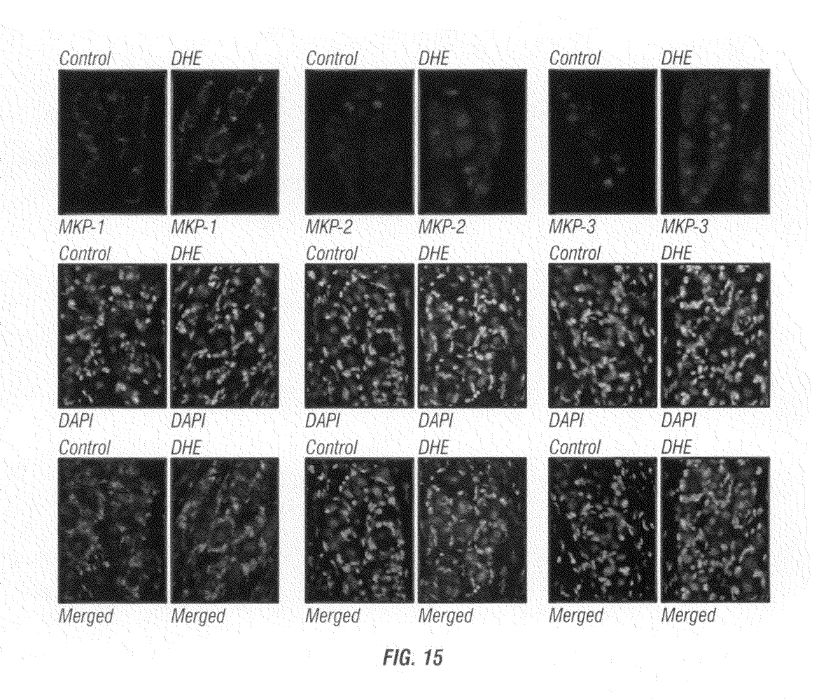

MAP kinase phosphatase levels were monitored in control subject, subjects treated with capsaicin, and subjects treated with DHE and capsaicin together. The results, as shown in FIGS. 10, 11, and 15, suggest that DHE modulates the MAP kinase signal transduction pathway by increasing MAP kinase phosphatase-1 (MKP-1), MKP-2, and MKP-3 levels (and activity) as well as repressing p38 MAP kinase 14 levels following stimulation by capsaicin.

Treatment of a subject experiencing a headache precursor event with DHE may have a protective effect upon the subject's neural, glial, and endothelial tissue via induction of MAP kinase phosphatase activity. The result can be pre-emption of a headache subsequent to treatment with DHE.

FIG. 16 illustrates a mechanism by which DHE may act to pre-empt a subsequent headache through suppression of cortical spreading depression which is presumed to occur during a headache precursor event, the subsequent secretion of CGRP and the onset of headache (particularly migraine headache). The inventors continue to not wish to be bound by a particular mechanism or hypothesis of action, although the Experimental evidence is supportive of efficacy in the aggregate.

Various triggering events headache precursor events can initiate Cortical Spreading Depression (CSD), a proposed initiating event for migraine pain, which results in the release of CGRP, kinins, and Substance P from the glia and endothelial cells. When these neurotransmitters effect the trigeminal nerve they cause pain and a second release of CGRP.

DHE, when administered during a headache precursor event, may exert its action via three mechanisms indicated in the red numbers in FIG. 16: (1) by interfering with the stimulus of the headache trigger so there is no CSD and thus no migraine pain; (2) even if a CSD occurs, DHE interferes with the resulting production of CGRPs, kinins, and Substance P; and (3) DHE interferes with the release of CGRPs from the trigeminal nerves.

DHE has been shown to particularly repress expression of CGRP, an inflammatory molecule produced by glia and neurons that can increase vasodilation of proximal blood vessels. As shown in FIG. 8, DHE represses release (secretion) of CGRP from the cells stimulated by KCl.

The invention will now be described in more detail.

Definitions

All publications, patents and patent applications cited herein, whether supra or infra, are hereby incorporated by reference in their entirety for all purposes.

As used herein and in the appended claims, the singular forms “a,” “an,” and “the” include plural reference unless the context clearly dictates otherwise. Thus, for example, a reference to “a particle” includes a plurality of such particles, and a reference to “a carrier” is a reference to one or more carriers and equivalents thereof, and so forth.

“Administering” or “administration” means dosing a pharmacologically active material, such as DHE, to a subject in a manner that is pharmacologically useful.

“Adolescent cluster headache” has the meaning ascribed in International Classification of Headache Disorders 2^{nd} Edition in Cephalalgia 24: Suppl 1:9-160 (2004).

“Adolescent migraine” has the meaning ascribed in International Classification of Headache Disorders 2^{nd} Edition in Cephalalgia 24: Suppl 1:9-160 (2004).

“Adult cluster headache” has the meaning ascribed in International Classification of Headache Disorders 2^{nd} Edition in Cephalalgia 24: Suppl 1:9-160 (2004).

“Adult migraine” has the meaning ascribed in International Classification of Headache Disorders 2^{nd} Edition in Cephalalgia 24: Suppl 1:9-160 (2004).

“Amount effective to pre-empt a subsequent headache in the subject” means the amount of drug necessary to achieve headache pre-emption in a typical subject.

“Chronic migraine” has the meaning ascribed in International Classification of Headache Disorders 2^{nd} Edition in Cephalalgia 24: Suppl 1:9-160 (2004).

“Cluster headache” has the meaning ascribed in International Classification of Headache Disorders 2^{nd} Edition in Cephalalgia 24: Suppl 1:9-160 (2004).

“Complex” means a reversible association of molecules, atoms, or ions through weak chemical bonds. DHE complexes are weak covalent, or noncovalent, ionically or non-ionically associated molecular level combinations of dihydroergotamine or pharmaceutically acceptable salts thereof with other molecules, for example: chelates, clathrates, PEGylation, protein and peptide, crown-ether and cyclodextrin associations.

“Dihydroergotamine” means the compound known generically as dihydroergotamine, having a chemical structure referred to as 5′α)-9,10-dihydro-12′-hydroxy-2′-methyl-5′-(phenylmethyl)-ergotaman-3′,6′,18-trione or alternatively using IUPAC nomenclature: (2R,4R,7R)-N-[(1S,2S,4R,7S)-7-benzyl-2-hydroxy-4-methyl-5,8-dioxo-3-oxa-6,9-diazatricyclo[7.3.0.0^{2,6}]dodecan-4-yl]-6-methyl-6,11-diazatetracyclo[7.6.1.0^{2,7}.0^{12,16}]hexadeca-1(16),9,12,14-tetraene-4-carboxamide. It has a molecular weight of 583.678 g/mol, and a chemical formula of C₃₃H₃₇N₅O₅. Dihydromergotamine may be used in the practice of this invention as the base, or as a pharmaceutically acceptable salt, or complex thereof (collectively “DHE”).

“Dosage form” means DHE in a medium, carrier, vehicle, or device suitable for administration to a subject. In embodiments of the present invention, preferred dosage forms comprise pressurized metered dose inhalers, breath actuated pressurized metered dose inhalers, dry powder inhalers, nebulizers including vibrating mesh, ultrasonic and jet nebulizers, soft mist inhalers, and vaporization/condensation dosage forms.

“Headache precursor event” means symptoms experienced by a subject in advance of suffering from a major headache, and is generally predictive of an upcoming headache. Headache precursor events can comprise prodrome symptoms, premonitory symptoms, aura prior to headache onset, initial headache in a headache cluster, and headache trigger events. Certain headache precursor events will now be discussed in more detail.

Prodrome symptoms are headache precursor events usually seen in migraine or cluster headache sufferers. They precede a severe headache by an interval ranging from less than an hour up to several days, preferably 1 to 24 hours prior to a severe headache such as a migraine or cluster headache. Prodrome symptoms include, but are not limited to changes in mood and sensatory capabilities, or visceral changes including the following:
1) Visual field changes such as, bright lights, zigzag lines, distortions in the size or shape of objects, vibrating visual field, scintillating scotoma, shimmering, pulsating patches, tunnel vision scotoma, blind or dark spots in the field of vision, curtain-like effect over one eye, slowly spreading spots or kaleidoscope effects on visual field;
2) Auditory changes such as auditory hallucinations, modification of voices or sounds in the environment, buzzing, tremolo, amplitude modulation or other modulations;
3) Strange smells (Phantosmia), saliva collecting in the mouth;
4) Feelings of numbness or tingling on one side of the face or body, feeling separated from one's body or as if the limbs are moving independently from the body, feeling as if one has to eat or go to the bathroom, anxiety or fear, weakness or unsteadiness; altered mood, irritability, depression or euphoria, fatigue, yawning, excessive sleepiness, craving for certain food; stiff muscles (especially in the neck),
5) Dimunition of mental acuity or alertness such as being unable to understand or comprehend spoken words during and after the aura or being unable to speak properly, despite the brain grasping what the person is trying to verbalize (Aphasia);
6) Nausea, constipation or diarrhea, increased urination, and other visceral symptoms.

Prodrome symptoms occur in approximately 40-60% of migrainuers. L Kelman “The Premonitory symptoms (prodrome): a tertiary care study of 893 migraineurs” Headache 44 (9): 865-72. (October 2004) (“Kelman”). There are no approved or proven therapeutic options for pre-empting a migraine by initiating therapy during prodrome. Triptans and DHE, which can abort an established headache, have also been tried during the prodrome to try and prevent a following headache. However, until the invention by the applicants, there was no good scientific data proving their efficacy. In fact, there is some data that suggests that 5HT1B/D receptors are not externalized and hence not available for triptans or DHE to act on till the onset of an actual headache. Even though use of intravenous DHE has been suggested and tried in the past during prodrome to prevent a subsequent headache, use of intravenous DHE is associated with a high incidence of nausea and other adverse events making this an unattractive option. J Olesen et al. eds., The Headaches (2^{nd} Edition) 464 (2000). As not all prodromes are followed by a headache, see Kelman above, inducing very uncomfortable adverse events in all patients experiencing prodrome is clinically very undesirable, and thus taught away from by the art.

Premonitory symptoms are headache precursor events usually seen in migraine or cluster headache sufferers. They precede a severe headache by an interval ranging from several days up to several weeks, preferably 1 to 4 weeks prior to a severe headache such as a migraine or cluster headache. See E Raimondi “Premonitory Symptoms in Cluster Headache” Current Pain and Headache Reports 5:55-59 (2001). Premonitory symptoms have been noted in the literature, and have been reported to include, but are not limited to, concentration problems, depression, food craving, physical hyperactivity, irritability, nausea, phonophobia, fatigue, sleep problems, stressed feeling, stiff neck and yawning. GG Schoonman et al., “The prevalence of premonitory symptoms in migraine: a questionnaire study in 461 patients” Cephalalgia 26: 1209-1213 (2006). There is considerable overlap between symptoms noted with prodrome and premonitory symptoms, and such symptoms may present in the same or nearly the same way. There are no approved or proven therapeutic options for pre-empting a migraine by initiating therapy during premonitory symptoms.

Aura prior to headache onset is a headache precursor event that is usually seen in 20-40% of headache sufferers, such as migraine sufferers. It can precede a headache by up to 36 hours, preferably up to 12 hours, more preferably up to 4 hours. Aura symptoms can be visual or sensory in nature. Visual symptoms comprise flashing lights, distortion of images, visual field abnormalities and distortion of color vision. Sensory symptoms comprise parasthesias and dysesthesias, along with other sensory symptoms. Aura symptoms can last up to an hour. There are no approved or proven therapeutic options for preventing a headache following the aura. At least one well controlled study has failed to demonstrate any efficacy of sumatriptan in preventing a headache when administered during the aura. There is some data which suggests that 5 HT1B/D receptors are not externalized and hence not available for triptans or DHE to act on till the onset of an actual headache. There is no approved drug that is indicated for pre-emption of headache by treatment during aura in advance of headache onset.

Initial headache in a headache cluster is a headache precursor event that is seen in cluster headache sufferers. Cluster headache is often seen in young men, and may exhibit a seasonal cyclicality. The headache usually is moderate to severe in intensity, wakes the patient up in the middle of the night, is usually unilateral, is associated with autonomic symptoms like tearing of the ipsilateral eye, Homers syndrome and redness. The headache lasts a few minutes up to a day, preferably 20 minutes up to an hour. In most patients this initial headache is followed by a series or “cluster” of several similar headaches in the next several days. There is no proven or approved therapy that can be used during the initial headache that has been demonstrated to prevent subsequent headaches. Injectable sumatriptan and oxygen inhalation have been used to abort the initial headache in a cluster once the headache has begun. However these treatments fail to abort or prevent occurrence of subsequent headaches. As injectable sumatriptan cannot prevent the onset of subsequent headache, many patients tend to treat each of subsequent headaches with additional doses of the same drug, exceeding the recommended maximum dose of the drug and potentially exposing themselves to serious adverse events.

Headache trigger is a headache precursor event that can initiate a migraine within minutes to hours of experiencing the trigger. Headaches can be triggered by certain smells, exposure to visual stimuli such as flickering lights or certain repetitive patterns, or other sensory stimuli. Headache triggers differ from migraine prodrome or aura in that migraine triggers will cause a migraine in a migraineur, whereas prodrome or aura are manifestations of a migraine that has already begun. There is no conventionally available therapy that can be used subsequent to the trigger that has been demonstrated to prevent a subsequent headache.

“Menstrual migraine” has the meaning ascribed in International Classification of Headache Disorders 2^{nd} Edition in Cephalalgia 24: Suppl 1:9-160 (2004).

“Migraine” has the meaning ascribed in International Classification of Headache Disorders 2^{nd} Edition in Cephalalgia 24: Suppl 1:9-160 (2004).

“Migraine with aura” has the meaning ascribed in International Classification of Headache Disorders 2^{nd} Edition in Cephalalgia 24: Suppl 1:9-160 (2004).

“Migraine without aura” has the meaning ascribed in International Classification of Headache Disorders 2^{nd} Edition in Cephalalgia 24: Suppl 1:9-160 (2004).

“Oral inhalation” means delivery of a drug, such as DHE, to the lung via inhalation through the mouth.

“Pediatric cluster headache” has the meaning ascribed in International Classification of Headache Disorders 2^{nd} Edition in Cephalalgia 24: Suppl 1:9-160 (2004).

“Pediatric migraine” has the meaning ascribed in International Classification of Headache Disorders 2^{nd} Edition in Cephalalgia 24: Suppl 1:9-160 (2004).

“Pharmaceutically acceptable salt” means any salt whose anion does not contribute significantly to the toxicity or pharmacological activity of the salt, and, as such, they are the pharmacological equivalents of the base of dihydroergotamine. Suitable pharmaceutically acceptable salts include acid addition salts which may, for example, be formed by reacting the drug compound with a suitable pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid.

Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate(embonate), palmitate, pantothenate, phosphate/diphosphate, polygalacturonate, salicylate, stearate, sulfate, subacetate, succinate, tannate, tartrate, teoclate, tosylate, triethiodide and valerate.

“Pre-empt a subsequent headache” or “subsequent headache pre-emption” means to avert clinical presentation of an oncoming headache, and its attendant clinical symptoms, prior to full clinical presentation of the headache.

“Subject” means an animal, including mammals such as humans and primates, that is the object of treatment or observation.

Formulation and Dosage Forms

Dosage forms according to the invention may comprise non-pharmacologically active ingredients such as, for example, buffers, tonicity agents, antioxidants and stabilizers, nonionic wetting or clarifying agents, viscosity-increasing agents, absorption enhancing agents, and the like.

Suitable absorption enhancement agents include N-acetylcysteine, polyethylene glycols, caffeine, cyclodextrin, glycerol, alkyl saccharides, lipids, lecithin, dimethylsulfoxide, and the like.

Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and preferably, between about pH 7 and pH 7.5. Suitable tonicity agents are dextran 40, dextran 70, dextrose, glycerin, potassium chloride, propylene glycol, sodium chloride, and the like, such that the sodium chloride equivalent of the ophthalmic solution is in the range 0.9 plus or minus 0.2%.

Suitable antioxidants and stabilizers include sodium bisulfite, sodium metabisulfite, sodium thiosulfite, thiourea, caffeine, chromoglycate salts, cyclodextrins and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxmethylpropylcellulose, lanolin, methylcellulose, petrolatum, polyethylene glycol, polyvinyl alcohol, polyvinylpyrrolidone, carboxymethylcellulose and the like.

In one embodiment, DHE is administered as an aerosol or a suspension directly to the lung epithelium, for example, using a nebulizer, atomizer, spray dispenser, inhaler, or the like. DHE may be administered to the alveolar epithelium, the bronchial epithelium, or both. In another embodiment, DHE is administered to the lung epithelium in the form of particles having a diameter of the range of about 0.05 to 20 μm. In a more preferred embodiment the particle diameter is of the range of between about 0.05 to 10 μm. In a yet more preferred embodiment the particle diameter is of the range of between about 0.4 to 3 μm.

A DHE powder useful in the present invention may be generated using a supercritical fluid processes. Supercritical fluid processes offer significant advantages in the production of DHE particles for inhalation delivery. Importantly, supercritical fluid processes produce respirable particles of the desired size in a single step, eliminating the need for secondary processes to reduce particle size. Therefore, the respirable particle produced using supercritical fluid processes have reduced surface free energy, which results in a decreased cohesive forces and reduced agglomeration. The particles produced also exhibit uniform size distribution. In addition, the particles produced have smooth surfaces and reproducible crystal structures which also tend to reduce agglomeration.

Such supercritical fluid processes may include rapid expansion (RES), solution enhanced diffusion (SEDS), gas-anti solvent (GAS), supercritical antisolvent (SAS), precipitation from gas-saturated solution (PGSS), precipitation with compressed antisolvent (PCA), aerosol solvent extraction system (ASES), or any combinations of the foregoing. The technology underlying each of these supercritical fluid processes is well known in the art and will not be repeated in this disclosure. In one specific embodiment, the supercritical fluid process used is the SEDS method as described by Palakodaty et al. in US Application 2003 0109421.

The supercritical fluid processes produce dry particulates that can be used directly by premetering into a dry powder inhaler (DPI) format, or the particulates may be suspended/dispersed directly into a suspending media, such as a pharmaceutically acceptable propellant, in a metered dose inhaler (MDI) format. The particles produced may be crystalline or may be amorphous depending on the supercritical fluid process used and the conditions employed (for example, the SEDS method is capable of producing amorphous particles). As discussed above, the particles produced have superior properties as compared to particles produced by traditional methods, including but not limited to, smooth, uniform surfaces, low energy, uniform particle size distribution and high purity. These characteristics enhance physicochemical stability of the particles and facilitate dispersion of the particles, when used in either DPI format or the MDI format.

The particle size should be such as to permit inhalation of the DHE particles into the lungs on administration of the aerosol particles. In one embodiment, the particle size distribution is less than 20 microns. In an alternate embodiment, the particle size distribution ranges from about 0.050 μm to 10.000 μm MMAD as measured by cascade impactors; in yet another alternate embodiment, the particle size distribution ranges from about and preferably between 0.4 and 3.5 μm MMAD as measured by cascade impactors.

The supercritical fluid processes discussed above produce particle sizes in the lower end of these ranges.

In the DPI format the DHE particles can be electrostatically, cryometrically, or traditionally metered into dosage forms as is known in the art. The DHE particle may be used alone (neat) or with one or more pharmaceutically acceptable excipients, such as carriers or dispersion powders including, but not limited to, lactose, mannose, maltose, etc., or surfactant coatings. In one preferred formulation, the DHE particles are used without additional excipients. One convenient dosage form commonly used in the art is the foil blister packs. In this embodiment, the DHE particles are metered into foil blister packs without additional excipients for use with a DPI. Typical doses metered can range from about 0.050 mg to 2 mg, or from about 0.250 mg to 0.500 mg. The blister packs are burst open and can be dispersed in the inhalation air by electrostatic, aerodynamic, or mechanical forces, or any combination thereof, as is known in the art. In one embodiment, more than 25% of the premetered dose will be delivered to the lungs upon inhalation; in an alternate embodiment, more 50% of the premetered dose will be delivered to the lungs upon inhalation; in yet another alternate embodiment, more than 80% of the premetered dose will be delivered to the lungs upon inhalation. The respirable fractions of DHE particles (as determined in accordance with the United States Pharmacopoeia, chapter 601) resulting from delivery in the DPI format range from 25% to 90%, with residual particles in the blister pack ranging from 5% or the premetered dose to 55% of the premetered dose.

In the MDI format the particles can be suspended/dispersed directly into a suspending media, such as a pharmaceutically acceptable propellant. In one particular embodiment, the suspending media is the propellant. It may be desirable that the propellant not serve as a solvent to the DHE particles. Suitable propellants include C₁₋₄ hydrofluoroalkane, such as, but not limited to 1,1,1,2-tetrafluoroethane (HFA 134a) and 1,1,1,2,3,3,3-heptafluoro-n-propane (HFA 227) either alone or in any combination. Carbon dioxide and alkanes, such as pentane, isopentane, butane, isobutane, propane and ethane, can also be used as propellants or blended with the C₁₋₄ hydrofluoroalkane propellants discussed above. In the case of blends, the propellant may contain from 0-25% of such carbon dioxide and 0-50% alkanes. In one embodiment, the DHE particulate dispersion is achieved without surfactants. In an alternate embodiment, the DHE particulate dispersion may contain surfactants if desired, with the surfactants present in mass ratios to the DHE ranging from 0.001 to 10. Typical surfactants include the oleates, stearates, myristates, alkylethers, alkylarylethers, sorbates and other surfactants used by those skilled in the art of formulating compounds for delivery by inhalation, or any combination of the foregoing. Specific surfactants include, but are not limited to, sorbitan monooleate (SPAN-80) and isopropyl myristate. The DHE particulate dispersion may also contain polar solvents in small amounts to aid in the solubilization of the surfactants, when used. Suitable polar compounds include C₂₋₆ alcohols and polyols, such as ethanol, isopropanol, polypropylene glycol and any combination of the foregoing. The polar compounds may be added at mass ratios to the propellant ranging from 0.0001% to 4%. Quantities of polar solvents in excess of 4% may react with the DHE or solubilize the DHE. In one particular embodiment, the polar compound is ethanol used at a mass ratio to the propellant from 0.0001 to 1%. No additional water or hydroxyl containing compounds are added to the DHE particle formulations other than is in equilibrium with pharmaceutically acceptable propellants and surfactants. The propellants and surfactants (if used) may be exposed to water of hydroxyl containing compounds prior to their use so that the water and hydroxyl containing compounds are at their equilibrium points. Standard metering valves (such as from Neotechnics, Valois, or Bespak) and canisters (such as from PressPart or Gemi) can be utilized as is appropriate for the propellant/surfactant composition. Canister fill volumes from 2.0 ml to 17 ml may be utilized to achieve dose counts from one (1) to several hundred actuations. A dose counter with lockout mechanism can optionally be provided to limit the specific dose count irrespective of the fill volume. The total mass of DHE in the propellant suspension will typically be in the range of 0.100 mg to 2.000 mg of DHE per 100 mcL of propellant.

An actuator with breath actuation can preferably be used to maximize inhalation coordination, but it is not mandatory to achieve therapeutic efficacy. The respirable fraction of such MDIs would range from 25% to 75% of the metered dose (as determined in accordance with the United States Pharmacopoeia, chapter 601).

A variety of dosage forms are useful in the practice of the invention, and are described in, for example, US Patent Application Number 2008/0118442. A few embodiments now will be discussed in more detail.

Dry Powder Inhalers

In a dry powder inhaler (DPI), the dose to be administered is stored in the form of a non-pressurized dry powder and, on actuation of the inhaler, the particles of the powder are inhaled by the subject. Similar to pressurized metered dose inhalers (pMDIs), a compressed gas may be used to dispense the powder. Alternatively, when the DPI is breath-actuated, the powder may be packaged in various forms, such as a loose powder, cake or pressed shape in a reservoir. Examples of these types of DPIs include the Turbohaler™ inhaler (Astrazeneca, Wilmington, Del.) and Clickhaler® inhaler (Innovata, Ruddington, Nottingham, UK). When a doctor blade or shutter slides across the powder, cake or shape, the powder is culled into a flowpath whereby the patient can inhale the powder in a single breath. Other powders are packaged as blisters, gelcaps, tabules, or other preformed vessels that may be pierced, crushed, or otherwise unsealed to release the powder into a flowpath for subsequent inhalation. Typical of these are the Diskus™ inhaler (Glaxo, Greenford, Middlesex, UK), EasyHaler® (Orion, Expoo, FI), and Novohaler™ inhalers. Still others release the powder into a chamber or capsule and use mechanical or electrical agitators to keep the drug suspended for a short period until the patient inhales. Examples of this are the Exubera® inhaler (Pfizer, New York, N.Y.), Qdose inhaler (Microdose, Monmouth Junction, N.J.), and Spiros® inhaler (Dura, San Diego, Calif.).

Pressurized Metered Dose Inhalers

pMDIs generally have two components: a canister in which the drug particles are stored under pressure in a suspension or solution form, and a receptacle used to hold and actuate the canister. The canister may contain multiple doses of the formulation, although it is possible to have single dose canisters as well. The canister may include a valve, typically a metering valve, from which the contents of the canister may be discharged. Aerosolized drug is dispensed from the pMDI by applying a force on the canister to push it into the receptacle, thereby opening the valve and causing the drug particles to be conveyed from the valve through the receptacle outlet. Upon discharge from the canister, the drug particles are atomized, forming an aerosol. PMDIs generally use propellants to pressurize the contents of the canister and to propel the drug particles out of the receptacle outlet. In pMDIs, the composition is provided in liquid form, and resides within the canister along with the propellant. The propellant may take a variety of forms. For example, the propellant may be a compressed gas or a liquefied gas. Chlorofluorocarbons (CFC) were once commonly used as liquid propellants, but have now been banned. They have been replaced by the now widely accepted hydrofluroralkane (HFA) propellants.

In some instances, a manual discharge of aerosolized drug must be coordinated with inhalation, so that the drug particles are entrained within the inspiratory air flow and conveyed to the lungs. In other instances, a breath-actuated trigger, such as that included in the Tempo® inhaler (MAP Pharmaceuticals, Mountain View, Calif.) may be employed that simultaneously discharges a dose of drug upon sensing inhalation, in other words, the device automatically discharges the drug aerosol when the user begins to inhale. These devices are known as breath-actuated pressurized metered dose inhalers (baMDIs).

Nebulizers

Nebulizers are liquid aerosol generators that convert bulk liquids, usually aqueous-based compositions, into mists or clouds of small droplets, having diameters less than 5 microns mass median aerodynamic diameter (MMAD), which can be inhaled into the lower respiratory tract. This process is called atomization. The bulk liquid contains particles of the therapeutic agent(s) or a solution of the therapeutic agent(s), and any necessary excipients. The droplets carry the therapeutic agent(s) into the nose, upper airways or deep lungs when the aerosol cloud is inhaled.

Pneumatic (jet) nebulizers use a pressurized gas supply as a driving force for liquid atomization. Compressed gas is delivered through a nozzle or jet to create a low pressure field which entrains a surrounding bulk liquid and shears it into a thin film or filaments. The film or filaments are unstable and break up into small droplets that are carried by the compressed gas flow into the inspiratory breath. Baffles inserted into the droplet plume screen out the larger droplets and return them to the bulk liquid reservoir. Examples include PARI LC Plus®, Sprint®, Devilbiss PulmoAide®, and Boehringer Ingelheim Respimat®.

Electromechanical nebulizers use electrically generated mechanical force to atomize liquids. The electromechanical driving force is applied by vibrating the bulk liquid at ultrasonic frequencies, or by forcing the bulk liquid through small holes in a thin film. The forces generate thin liquid films or filament streams which break up into small droplets to form a slow moving aerosol stream which can be entrained in an inspiratory flow.

One form of electromechanical nebulizers are ultrasonic nebulizers, in which the bulk liquid is coupled to a vibrator oscillating at frequencies in the ultrasonic range. The coupling is achieved by placing the liquid in direct contact with the vibrator such as a plate or ring in a holding cup, or by placing large droplets on a solid vibrating projector (a horn). The vibrations generate circular standing films which break up into droplets at their edges to atomize the liquid. Examples include DuroMist®, Drive Medical Beetle Neb®, Octive Tech Densylogic®, and John Bunn Nano-Sonic®.

Another form of an electromechanical nebulizer is a mesh nebulizer, in which the bulk liquid is driven through a mesh or membrane with small holes ranging from 2 to 8 microns in diameter, to generate thin filaments which immediately break up into small droplets. In certain designs, the liquid is forced through the mesh by applying pressure with a solenoid piston driver (AERx®), or by sandwiching the liquid between a piezoelectrically vibrated plate and the mesh, which results in a oscillatory pumping action (EFlow®, AerovectRx, TouchSpray™). In a second type the mesh vibrates back and forth through a standing column of the liquid to pump it through the holes (AeroNeb®). Examples include the AeroNeb Go®, Pro®; PARI EFlow®; Omron 22UE®; and Aradigm AERx®.

Typically, dosage forms according to the invention will be distributed, either to clinics, to physicians or to patients, in an administration kit, and the invention provides such a kit. Such kits comprise one or more of an administration device (e.g., inhalers, etc) and one or a plurality of doses or a reservoir or cache configured to deliver multiple doses of the composition as described above. In one embodiment, the dosage form is loaded with a DHE formulation. The kit can additionally comprise a carrier or diluent, a case, and instructions for employing the appropriate administration device. In some embodiments, an inhaler device is included. In one embodiment of this kit, the inhaler device is loaded with a reservoir containing a DHE formulation. In another embodiment the kit comprises one or more unit doses of the DHE formulation. In one embodiment, the inhaler device is a baMDI such the TEMPO™ Inhaler.

Methods of Administration

DHE may be administered according to the invention by oral inhalation using dosage forms such as those discussed elsewhere herein. Subjects may be experiencing a headache precursor event when DHE is administered according to the invention, or may have experienced a headache precursor event within a previous period. In embodiments, the previous period comprises 4 weeks, preferably the previous period comprises 1 week, more preferably the previous period comprises 1 day, and still more preferably the previous period comprises 1 hour. An advantage of being able to administer DHE in cases where a subject has experienced a headache precursor event within a previous period is that a subject may not always have recognized that a headache precursor event occurred until the event has ended. Thus there is still an opportunity to pre-empt a subsequent headache even after the headache precursor event has ended.

In embodiments, a delivered dose of DHE ranges from 0.0001 to 0.5 mg/kg per day, preferably from 0.0015 to 0.085 mg/kg per day.

In an embodiment, DHE is administered as a solution comprising about 0.01% to about 0.5% DHE. More preferably, the solution is a physiological saline solution. Preferably, the amount of solution administered is about 0.1 ml (0.5 mg) to about 5 ml @1mg/ml, depending on, for example, the concentration of the active ingredient. More preferably, the amount of solution is about 2.5-5 ml and is delivered as a suspension using a metered dose inhaler.

In embodiments, DHE is administered by oral inhalation at a rate such that the Cₘₐₓ per administration (typically two doses, or alternatively one dose, depending on the nature of the dosage form used) is less than 5,000, 10,000, 20,000, 30,000, 40,000, 50,000, or 60,000 pg/ml concentration in plasma in humans. The time following administration when the peak plasma concentration of DHE is attained (Tₘₐₓ) occurs within 10, 15, 20, 30, 45 or 60 minutes after administration.

In embodiments, oral inhalation of DHE results in Cₘₐₓ per administration (typically two doses, or alternatively one dose, depending on the nature of the dosage form used) of 8-hydroxy dihydroergotamine, at less than 5,000, 10,000, 20,000, 30,000, 40,000, 50,000, 60,000, 100,000 or 200,000 pg/ml. The Tₘₐₓ of 8-hydroxy dihydroergotamine is less than 30, 45, 60, 90, or 120 minutes after administration.

Administration may occur upon a subject's noticing the onset of a headache precursor event, or may rely on an objective measurement (such as a test of visual or mental acuity) of the onset of a headache precursor event.

### EXAMPLES

The invention will be more readily understood by reference to the following examples, which are included merely for purposes of illustration of certain aspects and embodiments of the present invention and not as limitations.

Those skilled in the art will appreciate that various adaptations and modifications of the just-described embodiments can be configured without departing from the scope and spirit of the invention. Other suitable techniques and methods known in the art can be applied in numerous specific modalities by one skilled in the art and in light of the description of the present invention described herein.

Therefore, it is to be understood that the invention can be practiced other than as specifically described herein. The above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

### Example 1

### Pharmacokinetic Profiles of DHE

DHE was administered to human subjects by intravenous and oral inhalation routes of administration.

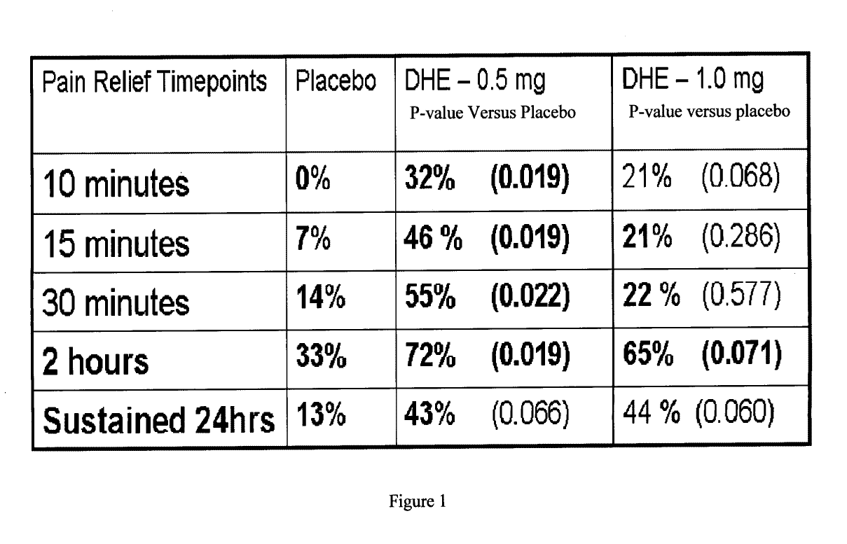

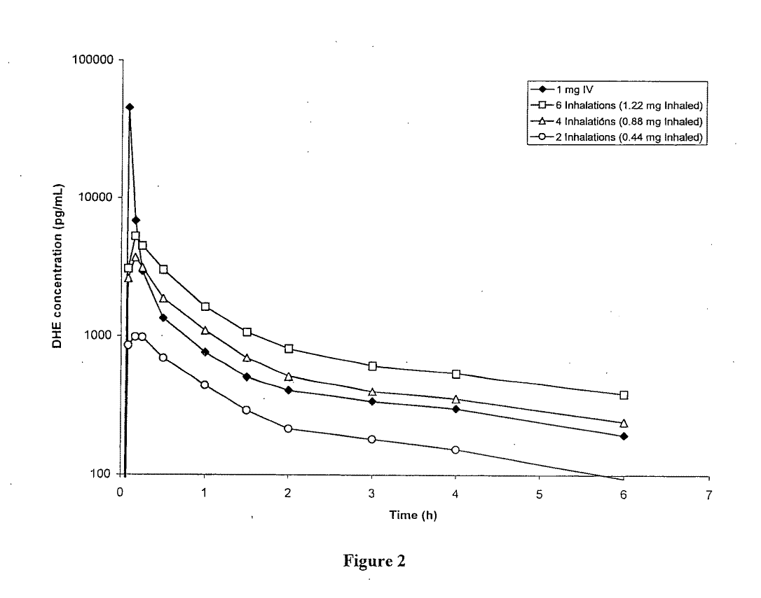

FIG. 1 shows the rapid pain relief (within 10 minutes) achieved by administering DHE by a method that achieves the two lower peak plasma concentration profiles shown in FIG. 2.

FIG. 2 shows DHE plasma profiles for 1 mg IV-administered DHE, compared to 6 inhalations (1.22 mg inhaled/fine particle dose), 4 inhalations (0.88 mg inhaled/fine particle dose) and 2 inhalations (0.44 mg inhaled/fine particle dose) of DHE respectively using a TEMPO® breath-actuated metered dose inahaler. A large plasma spike was observed following IV DHE administration, but not with inhaled delivery of DHE. This plasma spike difference (of at least 10×) may be associated with the reduced side effect profile, despite smaller differences in AUC between 1 mg IV and 0.88 mg inhaled DHE.

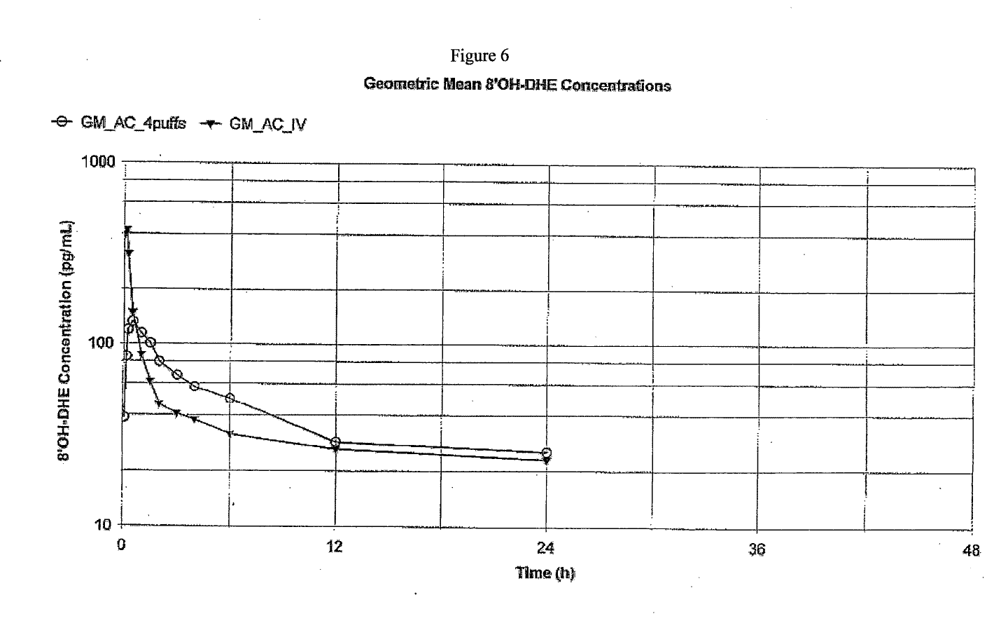

FIG. 6 shows the plasma profile of the primary metabolite of DHE, 8′-OH Dihydroergotamine, following intravenous and oral inhalation delivery of DHE. A larger plasma spike in 8′-OH Dihydroergotamine was observed following IV DHE administration, but not with inhaled delivery of DHE. This plasma spike difference also is hypothesized to be associated with the reduced side effect profile. The inhalable administration results in a peak plasma concentration of 8-hydroxy-dihydroergotamine of less than 1,000 pg/ml, preferably less than 500 pg/mL, more preferably less than 200 pg/mL at Cₘₐₓ in the circulating plasma. The inhalable administration also results in the Tₘₐₓ of the primary metabolites (e.g., 8′-OH Dihydroergotamine) to be less than 90 minutes in the circulating plasma.

The inventors have discovered that these slightly delayed, lower peak pharmacokinetic profiles are associated with minimized side effects. The side effects elicited by these administration profiles are shown in Table 1. The two lower curves, 0.88 mg and 0.44 mg DHE in FIG. 2, achieved therapeutic efficacy within 30 minutes, but elicited only minor side effects with the 0.88 mg dose, and no side effects were observed with the 0.44 mg dose. The highest curve, 1.0 mg IV DHE—the typical therapeutic regimen practiced in clinics today-resulted in significant side effects including nausea and emesis. The observed lower Cₘₐₓ or peak plasma concentration difference which was approximately 10 times lower than IV, was theorized to be associated with the observed differential side effect profile, while the smaller differences in AUC, differences of only 1.2×, between 1 mg IV and 0.88 mg inhaled enabled therapeutic efficacy.

TABLE 1
Side effects associated with the pharmacokinetic profiles in FIG. 2

| | 1 mg DIIE IV, | 0.88 mg DIIE |
|---|---|---|
| | n = 16 (%) | Inhaled, n = 12 (%) |
| Nervous System | | |
| Dizziness | 7 (44) 7r | 1(8) |
| Paresthesia | 5 (31) 5r | 0 |
| Gastrointestinal | | |
| System | | |
| Nausea | 10 (63) 10r | 1(8) |
| Vomiting | 2 (13) 2r | 0 |
| General disorders | | |
| Feeling hot | 3 (19) 3r | 0 |

r = considered by investigator related to study drug

### Example 2

### Pharmacokinetic Studies

A differential adverse effect profile was reported in a clinical study comparing 1 mg IV-administered DHE with inhaled DHE (Table 1). A greater incidence of adverse effects were apparent following IV dosing. To investigate pharmacologically-mediated adverse effect differences between (1) intravenous and (2) inhaled Dihydroergotamine Mesylate (DHE), biogenic amine receptor binding (serotonin (5-HT), adrenergic, dopaminergic) of dihydroergotamine mesylate in vitro was determined, based on concentrations corresponding to the Cₘₐₓ levels reported following inhaled and intravenous (IV) dosing in a clinical study.

To investigate the unexpected result that the lower spikes of DHE may have resulted in a different receptor binding profile thus achieving efficacy, but avoiding side effects, a clinical investigation of receptor binding at the Cₘₐₓ concentrations were undertaken.

Peak Plasma DHE concentrations (Cₘₐₓ) were determined from plasma samples (LC-MS/MS) following intravenous administration (1 mg) by infusion over 3 minutes, and from plasma samples (LC-MS/MS) following inhaled dosing (0.88 mg and 0.44 mg doses), where doses were given by multiple actuations from an inhaler over a period of 2-4 minutes. The inhaled doses represent the expected systemic delivered dose and were estimated from the fine particle dose delivered ex-actuator. The observed Cₘₐₓ data is presented in FIG. 2 for DHE. A similar approach was also taken with the primary metabolite, 8′-OH-DHE.

Table 2 presents in vitro concentrations equivalent to Cₘₐₓ. These concentrations were selected for receptor-binding investigations for both DHE and 8′-OH-DHE.

TABLE 2
Concentrations equivalent to peak plasma concentrations
investigated for receptor binding.

| | Dihydroergotamine | 8′-OH Dihydroergo- |
|---|---|---|
| Dose level | Mesylate (pg/mL) | tamine (pg/mL) |
| 1 mg IV | 53,215 | 378 |
| 0.88 mg | 4,287 | 149 |
| inhaled | | |
| 0.44 mg | 1,345 | 58 |
| inhaled | | |

### Example 3

### Serotonin, Adrenergic and Dopaminergic Receptor Binding by DHE at Concentrations Equivalent to Peak Plasma Concentrations

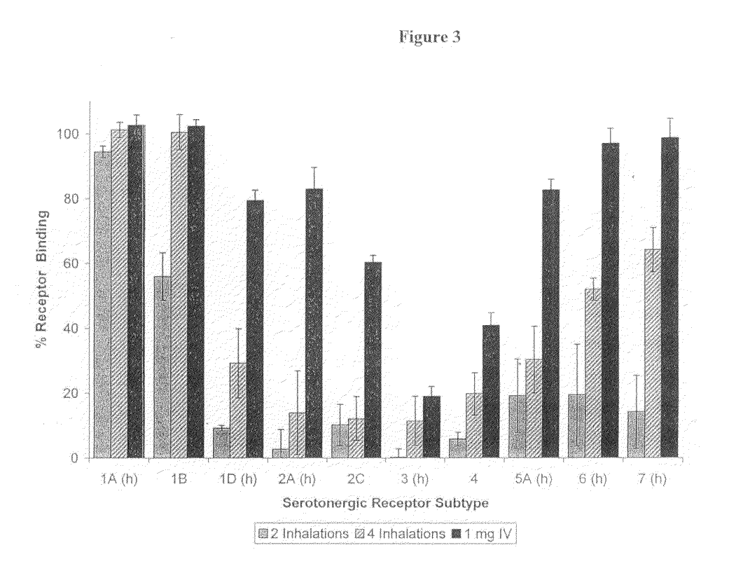

Radioligand receptor binding assays clearly show that DHE exhibits wide ranging pharmacology at multiple receptor sites. (FIGS. 3-5.) For the majority of receptors, DHE achieves significant binding at concentrations equivalent to the IV Cₘₐₓ whereas inhaled binding at each dose yields a different profile. In most instances, binding is reduced when non-IV methods are used to administer. The anti-migraine efficacy of DHE is due to agonist activity at 5-HT_{1B} and 5-HT_{1D} receptors. FIG. 3 shows receptor binding data at various serotonergic receptor subtypes, indicating greater response at several subtypes for intravenous administration at Cₘₐₓ. The notation “(h)” represents cloned human receptor subtypes. Similar trends were observed for adrenergic and dopaminergic subtypes. Binding at these receptors is demonstrated with 100% binding at 5-HT_{1B} following both 1 mg intravenous and 0.88 mg inhaled dosing. (FIG. 3.) Following inhalation, however, apparent binding at 5-HT_{1D} receptors is lower than IV. The long duration of DHE in circulation beyond Cₘₐₓ likely is due to biphasic elimination. (Wyss, P. A., Rosenthaler, J., Nuesch, E., Aellig, W. H. Pharmacokinetic investigation of oral and IV dihydroergotamine in healthy subjects. Eur. J. Clin. Pharmacol. 1991; 41:597-602). These results suggest that maximal receptor binding is not entirely necessary for the duration of clinical response.

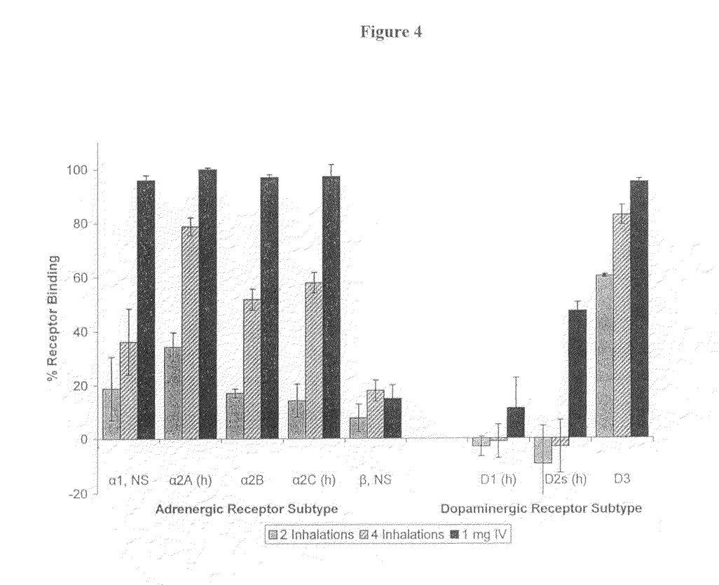

As seen in FIGS. 3-5, the IV method of administration with the high Cₘₐₓ which resulted in side effects, showed extensive binding at the dopaminergic and adrenergic receptors at concentrations equivalent to the peak plasma spikes (Cₘₐₓ) resulting from the IV administration method. FIG. 4 shows receptor binding data at adrenergic (left panel) and dopaminergic (right panel) receptors, indicating greater response at several subtypes for intravenous administration at Cₘₐₓ. The notation “(h)” represents cloned human receptor subtypes and “NS” indicates non-specific binding.

The dopaminergic receptors D1 and D2 are primarily responsible for nausea and emesis. Concentrations equivalent to the peak plasma spikes (Cₘₐₓ) resulting from the novel administration method that dampened and delayed the peak, as shown in FIG. 2, significantly lowered dopaminergic receptor binding, specifically at D2 and D1, as shown in FIG. 4, with the ultimate result of reducing nausea and emesis in the patients.

Similarly the lowered adrenergic binding shown in FIG. 4, corresponded to less vasoconstriction and lowered blood pressure or cardiovascular excursions in the patients. While receptor binding at the adrenergic and dopaminergic receptors were lower at concentrations equivalent to the peak plasma spikes (Cₘₐₓ) resulting from the novel administration method, the binding achieved by these administration methods at the serotonin receptors, specifically 5HT_{1a/d} was sufficient to be efficacious for treatment of migraine. (FIG. 3.)

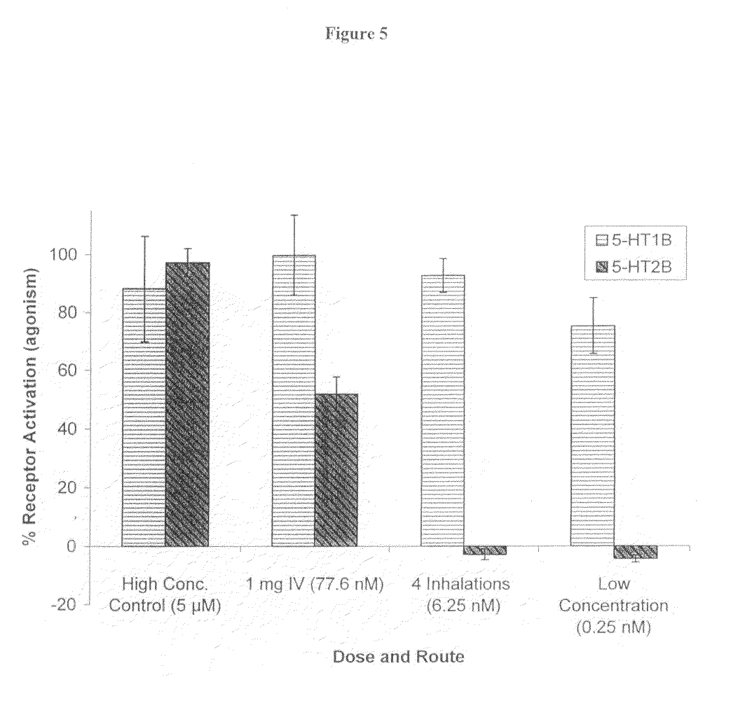

Agonists of 5-HT_{1B} subtype receptors are known to be useful in the treatment of migraine and associated symptoms. 5-HT_{2B} receptors are known to play a triggering role in the onset of migraine. FIG. 5 shows selective agonism at 5-HT_{1B} and 5-HT_{2B} receptors following high concentration control (5 μm), IV at Cₘₐₓ (77.6 nM), 4 inhalations at Cₘₐₓ (6.25 nM) and at a markedly reduced concentration (0.25 nM). Whereas 5-HT_{1B} agonism is maintained across all concentrations, indicating high potency, agonism is absent for orally-inhaled DHE at the 5-HT_{2B} receptors.

It is noted that all three methods of administration achieve rapid plasma levels within 20 minutes, with concentrations sufficient to bind the serotonin receptors and effect rapid treatment of migraine. (FIG. 2).

### Example 4

### Pulmonary Administration of DHE Formulations Using a TEMPO™ Inhaler

DHE powder is generated using supercritical fluid processes that produce respirable particles of the desired size in a single step. (see WO2005/025506A2.)

A controlled particle size for the microcrystals was chosen to ensure that a significant fraction of DHE would be deposited in the lung.

A blend of two inert and non-flammable HFA propellants were selected as part of formulation development) for the drug product: HFA 134a (1,1,1,2-tetrafluoroethane) and HFA 227ea (1,1,1,2,3,3,3-heptafluoropropane). The finished product contained a propellant blend of 70:30 HFA 227ea:HFA 134a, which was matched to the density of DHE crystals in order to promote pMDI suspension physical stability. The resultant suspension did not sediment or cream (which can precipitate irreversible agglomeration) and instead existed as a suspended loosely flocculated system, which is easily dispersed when shaken. Loosely fluctuated systems are well regarded to provide optimal stability for pMDI canisters. As a result of the formulation's properties, the formulation contained no ethanol and no surfactants/stabilizing agents.

The DHE formulation was administered to patients using TEMPO™, a novel breath activated metered dose inhaler. TEMPO™ overcomes the variability associated with standard pressurized metered dose inhalers (pMDI), and achieve consistent delivery of drug to the lung periphery where it can be systemically absorbed. To do so, TEMPO™ incorporates four novel features: 1) breath synchronous trigger—can be adjusted for different drugs and target populations to deliver the drug at a specific part of the inspiratory cycle, 2) plume control—an impinging jet to slow down the aerosol plume within the actuator, 3) vortexing chamber—consisting of porous wall, which provides an air cushion to keep the slowed aerosol plume suspended and air inlets on the back wall which drive the slowed aerosol plume into a vortex pattern, maintaining the aerosol in suspension and allowing the particle size to reduce as the HFA propellant evaporates, and 4) dose counter—will determine the doses remaining and prevent more than the intended maximum dose to be administered from any one canister. Features 2 and 3 have been shown to dramatically slow the deposition and improve lung deposition of the Emitted Dose (ED), by boosting the Fine Particle Fraction (FPF).

### Example 5

### DHE Suppresses Secretion of Inflammatory Molecules In Vitro

These experiments investigated the cellular events within trigeminal ganglia that may account for the therapeutic benefit of DHE in the pre-emptive treatment of migraine and cluster headache.

Trigeminal ganglia comprise ˜10% neurons, ˜90% glia, and ˜2% Schwann cells. They are located in the mammalian head, usually posterior and adjacent to the orbit.

Primary trigeminal ganglion cultures were established using trigeminal ganglia dissected from day 2-3 (2-3 PN) neonate Sprague Dawley rats. Cultures were maintained for 1 d and were then untreated (control), treated 1 h with 60 mM KCl, 1 h with 2 μM capsaicin, 1 h with 1 μM or 10 μM DHE, 1 h with 1 μM or 10 μM Sumatriptan, or pretreated with DHE or Sumatriptan for 30 minutes prior to addition of stimulatory agents.

The amount of CGRP released into the culture medium was determined by radioimmunoassay and normalized to total protein as determined using the modified method of Bradford (Bradford (1976) Anal. Biochem. 72: 248-254). Statistical significance was determined using Mann-Whitney U non-parametric test. Differences considered statistically significant at p<0.05. Cultured cells were also stained for protein expression of β-tubulin, CGRP, and 5-HT₁ receptors using specific antibodies (Abs) and immunohistochemistry.

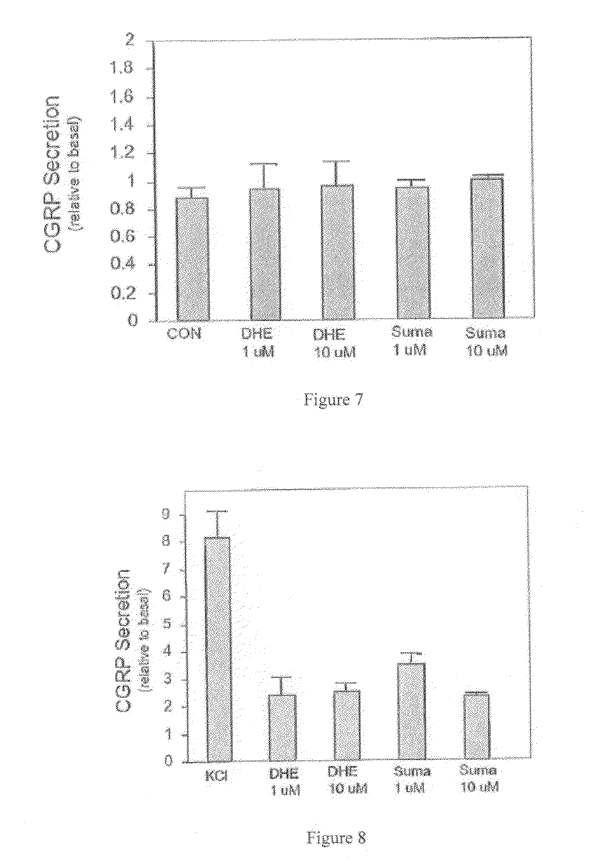

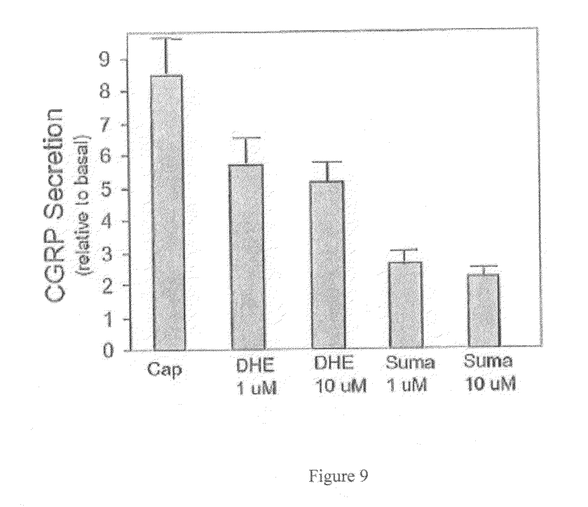

FIG. 7 shows that DHE or Sumatriptan (Suma) had no apparent effect upon basal secretion of CGRP into the medium. However, stimulation of the culture using KCl was reduced in the presence of DHE and Suma by approximately 68% and 70%, respectively (see FIG. 8). In addition, stimulation of the culture using capsaicin was reduced in the presence of DHE and Suma by approximately 38% and 71%, respectively (see FIG. 9).

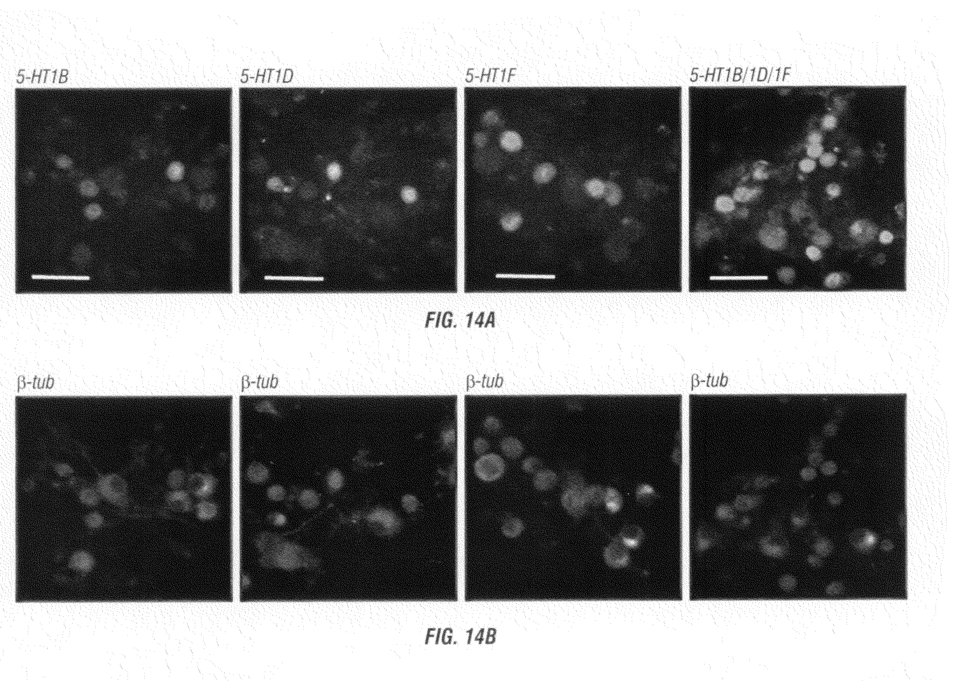

FIGS. 14A and B show typical results for immunohistochemical staining using Abs against β-tubulin, CGRP, and 5-HT₁ receptors. The results show that the expression of CGRP and 5-HT₁ receptors co-localized with the cells and with β-tubulin.

### Example 6

### DHE Suppresses Secretion of Inflammatory Molecules In Vivo

Adult (A) Sprague Dawley rats were anaesthetized by intraperitoneal (i.p.) injection of 0.3 ml ketamine and xylazine (Sigma Chemical Co. St. Louis, Mo.; 800 mg and 60 mg per 10 ml, respectively). The animals were then injected in the eyebrow region with 10 μM capsaicin for 2 h, 10 mg/kg DHE i.p. for 1 h, or were pretreated with DHE for 1 h prior to injection with capsaicin. Trigeminal ganglia were collected and placed in optimal cutting temperature (OCT) prior to cryosectioning. Sections were then stained using antibodies for CGRP and MKPs.

As shown in FIG. 10, treatment with DHE resulted in an increase of MAP kinase phosphate-1 levels by at least 10% in the trigeminal ganglial neurones and satellite glia. Similar results were obtained in separate experiments to determine levels of MKP-1, MKP-2, and MKP-3 following treatment with DHE.

FIG. 11 shows that treatment with DHE also repressed capsaicin-induced expression of p38 MAP kinase 14.

FIG. 12, in contrast, shows that DHE repressed capsaicin-induced diffusion of TRUEBLUE dye between neurons and glia at least 10%%. FIG. 13 shows that levels of connexin 26, a gap-junction component protein, are also repressed following treatment with DHE.

Primary trigeminal ganglion cultures or 20 μm sections of trigeminal ganglia were fixed in 4% paraformaldehyde, stained with antibodies for CGRP (Neuromics, 1:500), β-tubulin (Sigma, 1:1000), 5-HT₁ receptors (Santa Cruz, 1:100), MKP-1 (Upstate, 1:500), MKP-2 (Santa Cruz, 1:500), or MKP-3 (Santa Cruz, 1:500). Immunoreactive proteins were visualized using rhodamine Red-X-conjugated (β-tubulin and MKPs) or FITC-conjugated (5-HT₁ and CGRP) secondary antibodies (1:100 dilution in PBS, Jackson ImmunoResearch Laboratories).

What is claimed is:

1. A method comprising:
providing a subject experiencing a headache precursor event;
administering dihydroergotamine, or a pharmaceutically acceptable salt or complex thereof, to the subject by oral inhalation, in an amount effective to pre-empt a subsequent headache in the subject.

2. The method of claim 1, wherein the pharmaceutically acceptable salt of dihydroergotamine comprises acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate(embonate), palmitate, pantothenate, phosphate/diphosphate, polygalacturonate, salicylate, stearate, sulfate, subacetate, succinate, tannate, tartrate, teoclate, tosylate, triethiodide or valerate.

3. The method of claim 2, wherein the pharmaceutically acceptable salt comprises a mesylate salt of dihydroergotamine.

4. The method of claim 1, wherein the dihydroergotamine, or a pharmaceutically acceptable salt or complex thereof, is administered to the subject by oral inhalation using a dosage form that comprises a pressurized metered dose inhaler, breath actuated pressurized metered dose inhaler, dry powder inhaler, or a nebulizer.

5. The method of claim 1, wherein the amount effective to pre-empt a subsequent headache in the subject comprises a delivered dose of dihydroergotamine, or a pharmaceutically acceptable salt or complex thereof, that ranges from 0.0001 to 0.5 mg/kg per day.

6. The method of claim 5, wherein the delivered dose of dihydroergotamine, or a pharmaceutically acceptable salt or complex thereof, ranges from 0.0015 to 0.085 mg/kg per day.

7. The method of claim 1, wherein the headache comprises migraine or cluster headache.

8. The method of claim 7, wherein the migraine comprises migraine with aura, migraine without aura, chronic migraine, pediatric migraine, adolescent migraine, adult migraine, or menstrual migraine.

9. The method of claim 7, wherein the cluster headache comprises pediatric cluster headache, adolescent cluster headache, or adult cluster headache.

10. The method of claim 1, wherein the headache precursor event comprises prodrome symptoms, premonitory symptoms, aura prior to headache onset, initial headache in a headache cluster, or a headache trigger event.

11. A method comprising:
providing a subject experiencing a headache precursor event, or who has experienced a headache precursor event within a previous period;
administering dihydroergotamine, or a pharmaceutically acceptable salt or complex thereof, to the subject by oral inhalation, in an amount effective to pre-empt a subsequent headache in the subject.

12. The method of claim 12, wherein the previous period comprises 4 weeks.

13. The method of claim 12, wherein the previous period comprises 1 week.

14. The method of claim 13, wherein the previous period comprises 1 day.

15. The method of claim 14, wherein the previous period comprises 1 hour.
